(19) United States
(12) Patent Application Publication
(10) Pub. No.: US 2015/0190572 A1
(43) Pub. Date: Jul. 9, 2015

(54) Trace Control System for an Intravenous Drip

(71) Applicant: Yi-Ping Tzeng, Pingtung City (TW)

(72) Inventor: Yi-Ping Tzeng, Pingtung City (TW)

(21) Appl. No.: 14/580,306

(22) Filed: Dec. 23, 2014

(30) Foreign Application Priority Data
Jan. 7, 2014 (TW) 103200268

(51) Int. Cl. A61M 5/168; A61M 5/142

(52) U.S. Cl. CPC A61M 5/16813; A61M 5/142; A61M 2005/14208

(57) ABSTRACT

A trace control system for an intravenous drip includes a housing having an inlet and an outlet. A cover is mounted to a side of the housing. The cover and the housing together define a receiving space. The inlet and the outlet are provided on an outer periphery of the housing and intercommunicate with the receiving space. A shaft unit is received in the receiving space. An impeller is coupled to the shaft unit and includes at least one vane. At least two magnetic elements are provided on the at least one vane. A magnetically inductive actuator is mounted to the outer periphery of the housing and detects the at least two magnetic elements. A controller is coupled to the magnetically inductive actuator and controls operation timing of the magnetically inductive actuator.

### BACKGROUND OF THE INVENTION

1. Field of the Invention

The present invention relates to a trace control system and, more particularly, to a trace control system connected to an intravenous drip bag for simply, conveniently, and precisely controlling liquid delivery.

2. Description of the Related Art

The amount of a liquid in an intravenous drip bag to be injected into a human body during a medical procedure must be calculated carefully to avoid hazards resulting from overdose or insufficient dose. A paramedic often has to take care of about 10-20 sickbeds due to severe shortage of paramedics and often has to work more than ten hours, leading to frequent medical disputes. Intravenous drips are one of the most frequently neglected medical procedures and take considerable time.

A paramedic must have to ensure whether the intravenous drip bag is empty and have to be aware of the flow rate and time of the intravenous fluid during an intravenous drip. The paramedic is further burdened if the intravenous administration is intermittent. Generally, the flow rate of the intravenous fluid in an intravenous drip can be adjusted, but the paramedic cannot be reminded of finish of the intravenous drip. Furthermore, the dose cannot be set automatically. Improvement is, thus, required.

FIG. 1 shows a peristaltic pump 9 (also referred to as an intravenous pump) currently available in the market for fixed-dose administration. In operation, when the peristaltic pump 9 is driven by an external force to rotate, a hose 91 is squeezed by rollers 92 and deforms. The pressure is released at an outlet 93 of the hose 91 to output the liquid in the hose 91. However, the peristaltic pump 9 has the following disadvantages:

1. When the pressure accumulated in the compressed hose 91 is released, a pulsed pressure is generated and causes discomfort to a body of a human receiving the liquid.

2. The dose is limited by the diameter of the hose 91 and the compression space, resulting in limitation to the control tolerance of the dose.

3. The hose 91 is apt to break by squeezing and, thus, has a short service life.

4. The whole system is complicated in structure and is expensive.

5. The system requires a motor as the power source, leading to an increase in the costs.

6. The hose 91 is made of a special material and cannot be replaced by currently available medical hoses, leading to an increase in the consumables.

The capacities of intravenous drip bags are generally 250 ml, 400 ml, 500 ml, or 1000 ml. However, the liquid in an intravenous drip bag is not completely delivered every time, and non-continuous delivery is not always the best solution to the patient. Thus, it would be commercially valuable and contributive to the medical society if a specific amount of liquid can be delivered within a specific period of time while overcoming the drawbacks of the conventional peristaltic pump 9.

Furthermore, in a case that the amount of liquid to be delivered is 450 ml in a description from a doctor, a paramedic would release 50 ml of liquid out of a 500 ml intravenous drip bag, which causes a waste of medicine and increases the operation time.

Taiwan Utility Model No. M404020 discloses an active type intravenous injection system including a housing, a flow control system, an intravenous bag, and a rotary pressing head. The flow control system includes a flow controller, a control cable, a peristaltic pump, and a power cord. The peristaltic pump can be operated to drive the rotary pressing head to rotate and squeeze a drip hose full of a liquid. The liquid in the drip hose continuously and quantitatively flows forward to achieve the demand of high-precision flow under cooperation with a low pulse pump head. However, use of the peristaltic pump incurs the above problems and drawbacks.

Thus, a need exists for a novel trace control system for an intravenous drip achieving higher values.

### SUMMARY OF THE INVENTION

The primary objective of the present invention is to provide a trace control system for an intravenous drip using magnetic driving as a power source to reduce the costs and to simplify the structure.

The secondary objective of the present invention is to provide a trace control system for an intravenous drip including replaceable vanes to change the volume of the liquid to be stored.

Another objective of the present invention is to provide a trace control system for intravenous drip capable of reducing the pulse pressure.

A further objective of the present invention is to provide a trace control system for intravenous drip that can rapidly be coupled with hoses of a standard specification used in hospitals to reduce the consumable expenses.

The present invention fulfills the above objectives by providing a trace control system for an intravenous drip including a housing having an inlet and an outlet. A cover is mounted to a side of the housing. The cover and the housing together define a receiving space. The inlet and the outlet are provided on an outer periphery of the housing and intercommunicate with the receiving space. A shaft unit is received in the receiving space. An impeller is coupled to the shaft unit and includes at least one vane. At least two magnetic elements are provided on the at least one vane. A magnetically inductive actuator is mounted to the outer periphery of the housing and detects the at least two magnetic elements. A controller is coupled to the magnetically inductive actuator and controls operation timing of the magnetically inductive actuator.

An end of the outlet is connected to the housing, and the other end the outlet can form a quick coupler. The outlet includes an inner periphery defining a liquid storage space.

An end of the inlet is connected to the housing, and the other end of the inlet can form a quick coupler.

The at least one vane can include a plurality of vanes. Each of the plurality of vanes includes a free end having an inclined guiding face. One of the at least two magnetic elements is provided on the inclined guiding face of the free end of one of the plurality of vanes.

In an example, the free end of each of the plurality of vanes further includes another face opposite to the inclined guiding face. Another of the at least two magnetic elements is provided on the another face of the free end of each of the plurality of vanes. The polarity of the magnetic element on each inclined guiding face is opposite to the polarity of the magnetic element on each another face.

The trace control system can further include another magnetically inductive actuator mounted on the outer periphery of the housing and diametrically opposed to the magnetically inductive actuator.

The shaft unit can include a shaft, a ball bearing, and a waterproof washer. The shaft is coupled with the ball bearing. The waterproof washer is mounted between the ball bearing and the housing.

The present invention will become clearer in light of the following detailed description of illustrative embodiments of this invention described in connection with the drawings.

### BRIEF DESCRIPTION OF THE DRAWINGS

FIG. 1 is a cross sectional view illustrating operation of a conventional peristaltic pump.

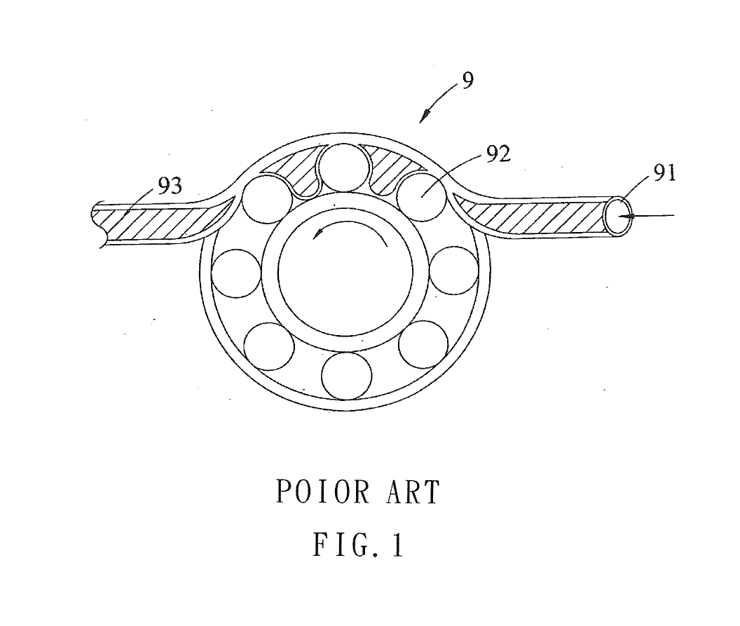

FIG. 2 is a perspective view of a trace control system for an intravenous drip of an embodiment according to the present invention.

FIG. 3 is an exploded, perspective view of the trace control system for an intravenous drip of the embodiment according to the present invention.

FIG. 4 an elevational view of a trace control system for an intravenous drip of another embodiment according to the present invention using two magnetically inductive actuators.

FIG. 5 is a cross sectional view illustrating operation of the trace control system for an intravenous drip of the embodiment according to the present invention.

### DETAILED DESCRIPTION OF THE INVENTION

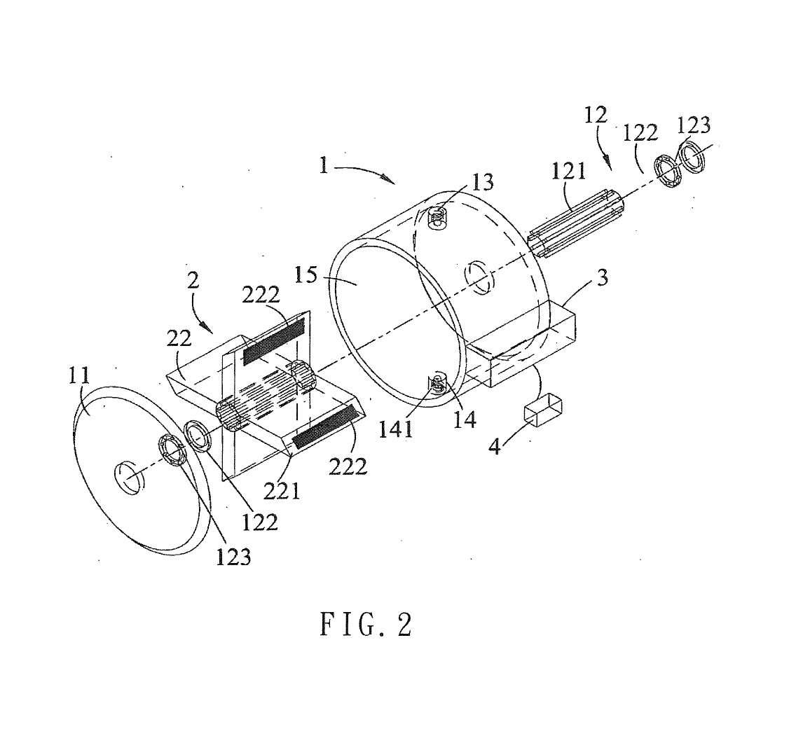

With reference to FIG. 2, a trace control system for an intravenous drip of an embodiment according to the present invention includes a housing 1, an impeller 2, a magnetically inductive actuator 3, and a controller 4. The impeller 2 is received in the housing 1. The magnetically inductive actuator 3 is mounted to an outer periphery of the housing 1. The controller 4 is electrically connected to the magnetically inductive actuator 3. In an example, the controller 4 is connected to the magnetically inductive actuator 3 by wireless connection. The controller 4 changes of the polarity of the magnetically inductive actuator 3 by electrical control to drive the impeller 2 in the housing 1 to rotate.

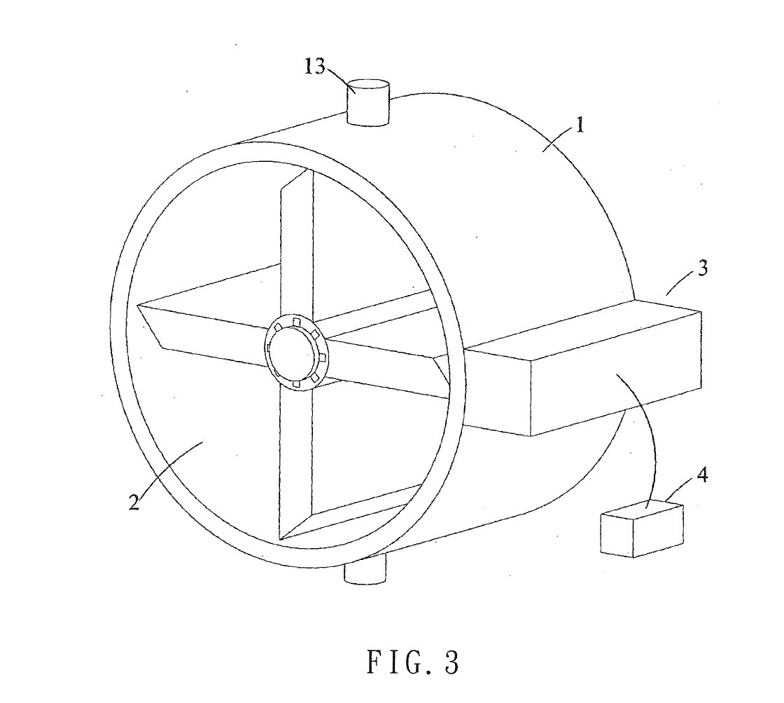

With reference to FIG. 3, the housing 1 includes an inlet 13 and an outlet 14. A cover 11 is mounted to a side of the housing 1. The cover 11 and the housing 1 together define a receiving space 15. The cover 11 can be made of a transparent material. The cover 11 can be coupled to the housing 1 by any suitable provision, including, but not limited to, male/female coupling, screwing, and force fitting. Furthermore, the cover 11 can be opened. Namely, the cover 11 can easily be moved between an open position and a closed position providing a sealing effect. A shaft unit 12 is received in the receiving space 15. The inlet 13 and the outlet 14 are provided on an outer periphery of the housing 1 and intercommunicate with the receiving space 15.

The shaft unit 12 includes a shaft 121, a ball bearing 122, and a waterproof washer 123. The shaft 121 includes an outer periphery having a plurality of grooves. An end of the shaft 121 is coupled with the ball bearing 122. The other end of the shaft 121 is a free end. The waterproof washer 123 is mounted between the ball bearing 122 and the housing 1 to prevent liquid leakage.

An end of the inlet 13 is connected to the housing 1. The other end of the inlet 13 forms a quick coupler that can be of a commercially available type.

An end of the outlet 14 is connected to the housing 1. The other end of the outlet 14 forms a quick coupler. The outlet 14 defines an interior space for storing a liquid. Specifically, the outlet 14 includes an inner periphery defining a liquid storage space 141. The liquid storage space 141 stores the liquid flowing from the receiving space 15.

An impeller 2 includes a coupling portion 21 and a plurality of vanes 22. The coupling portion 21 can be mounted to the shaft 121. Specifically, the coupling portion 21 can couple with the grooves of the shaft 121 such that the impeller 2 and the shaft 121 rotate jointly. The vanes 22 are located outside of the coupling portion 21. In this embodiment, the impeller 2 includes four vanes 22. An end of each vane 22 is connected to the outer periphery of the coupling portion 21. The other end of each vane 22 is a free end having an inclined guiding face 221. A magnetic element 222 is provided on each inclined guiding face 221, and another magnetic element 222 is provided on another face opposite to the inclined guiding face 221 and has a polarity opposite to a polarity of the magnetic element 222 on the inclined guiding face 221.

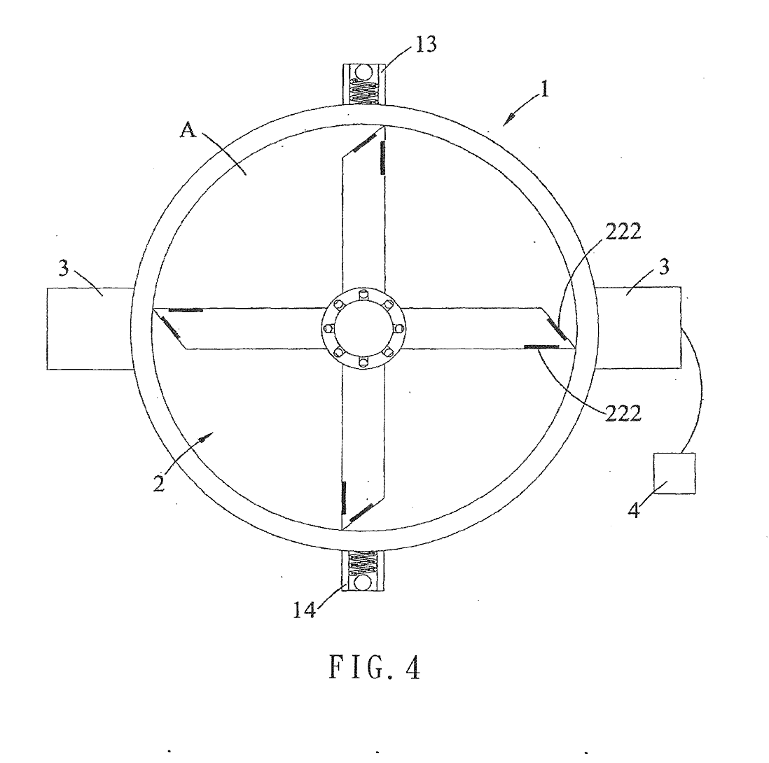

With reference to FIG. 4, an edge of the inclined guiding face 221 abuts an inner periphery of the housing 1 such that a chamber A is formed between two adjacent vanes 22 and the inner periphery of the housing 1. Each chamber A is a sealed space such that liquid in each chamber A will not leak into other chambers A.

The magnetically inductive actuator 3 is mounted on the outer periphery of the housing 1. The trace control system can include a plurality of magnetically inductive actuators 3. In an embodiment shown in FIG. 4, two magnetically inductive actuators 3 are mounted on the outer periphery of the housing 1 and are diametrically opposed to each other. If desired, more magnetically inductive actuator 3 can be provided and arranged in a symmetric manner. Each magnetically inductive actuator 3 can detect the magnetic elements 222. Specifically, each magnetically inductive actuator 3 can be a magnetic converter that can be electrified to control its polarity to become a north pole N or a south pole S. Detailed description of the magnetic converter is not required, because it is known in the art.

The controller 4 can be a central processing unit (CPU) having a control interface. The controller 4 can be coupled to the magnetically inductive actuator 3 by wire or wireless connection to thereby control the polarity of the magnetically inductive actuator 3. Furthermore, the magnetically inductive actuator 3 can send a signal indicative of the polarity of the magnetic element 222 detected by the magnetically inductive actuator 3. The number of the signals can be counted. Furthermore, the controller 4 can provide an active reminding function, such as providing a sound effect and/or a lighting effect triggered by the number of the signals. Furthermore, the controller 4 can be connected to a smart phone or a host at a nurse station to provide an active reminding effect.

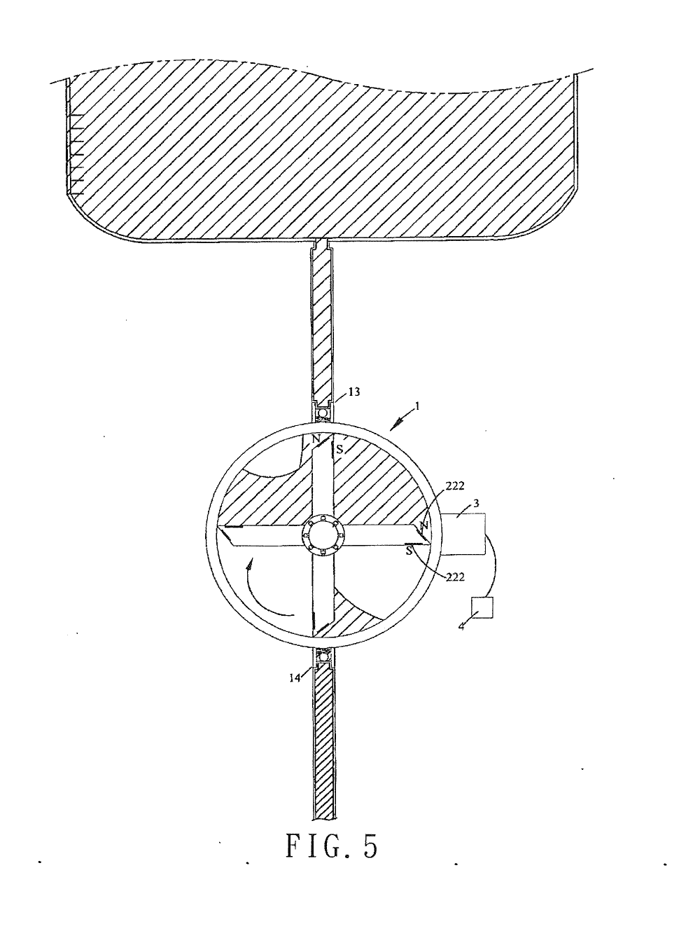

FIG. 5 shows operation of the trace control system for an intravenous drip according to the present invention. The liquid in an intravenous drip bag flows into the receiving space 15 via the inlet 13. Specifically, when the liquid fills one of the chambers A, the controller 4 controls the magnetically inductive actuator 3 to become a north pole N. A repulsive force is generated by the magnetically inductive actuator 3 and the magnetic element 222 aligned with the magnetically inductive actuator 3 having the same polarity as the magnetically inductive actuator 3. Thus, the vane 22 with this magnetic element 222 is moved away from the magnetically inductive actuator 3. Next, the controller 4 turns the magnetically inductive actuator 3 into a south pole S such that the magnetically inductive actuator 3 attracts the magnetic element 222 on the other face of the incoming vane 22. Thus, the incoming vane 22 moves faster towards the magnetically inductive actuator 3 under the attractive force, driving the impeller 2 to rotate. When the chamber A filled with the liquid intercommunicates with the outlet 14, the liquid gradually flows into the liquid storage space 141 and then into the body of a patient. Due to the buffering effect provided by the liquid storage space 141, the pulse pressure resulting from delivery of the liquid from the outlet 14 into the body of the patient can be reduced. Furthermore, the rotating speed and timing of the impeller 2 can be controlled by the controller 4 to provide a quantitative and regular administration effect. Furthermore, the vanes 22 are replaceable to permit easy cleaning and to permit replacement of an impeller 2 with a different number of vanes 22 such that the volume of each chamber A can be changed according to needs.

By using the magnetically inductive actuator 3 as a driving member, the structure of the trace control system for an intravenous drip according to the present invention can be simplified to reduce the costs. Furthermore, the impeller 2 is replaceable such that the chamber A can be varied to increase the control tolerance of the dose. The disadvantages resulting from squeezing a hose are avoided. Thus, the trace control system for an intravenous drip according to the present invention has a long service life, and the expenses of consumables can be reduced. Furthermore, the buffering effect provided by the liquid storage space 141 reduces the pulse pressure of the liquid delivered to the body of the patient, reducing the discomfort of the patient. Furthermore, the inlet 13 and the outlet 14 can include fast couplers for rapid coupling with hoses of a standard specification used in hospitals. The operation time and the consumable expenses are, thus, be reduced.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. A trace control system for an intravenous drip comprising:
a housing including an inlet and an outlet, with a cover mounted to a side of the housing, with the cover and the housing together defining a receiving space, with the inlet and the outlet provided on an outer periphery of the housing and intercommunicated with the receiving space, with a shaft unit received in the receiving space;
an impeller coupled to the shaft unit, with the impeller including at least one vane, with at least two magnetic elements provided on the at least one vane;
a magnetically inductive actuator mounted to the outer periphery of the housing, with the magnetically inductive actuator detecting the at least two magnetic elements; and
a controller coupled to the magnetically inductive actuator, with the controller controlling operation timing of the magnetically inductive actuator.

2. The trace control system for an intravenous drip as claimed in claim 1, with the outlet including an end connected to the housing, with the outlet further including another end forming a quick coupler, and with the outlet including an inner periphery defining a liquid storage space.

3. The trace control system for an intravenous drip as claimed in claim 1, with the inlet including an end connected to the housing, and with the inlet further including another end forming a quick coupler.

4. The trace control system for an intravenous drip as claimed in claim 1, with the at least one vane including a plurality of vanes, with each of the plurality of vanes including a free end having an inclined guiding face, and with one of the at least two magnetic elements provided on the inclined guiding face of the free end of each of the plurality of vanes.

5. The trace control system for an intravenous drip as claimed in claim 4, with the free end of each of the plurality of vanes further including another face opposite to the inclined guiding face, with another of the at least two magnetic elements provided on the another face of the free end of each of the plurality of vanes, and with a polarity of the magnetic element on each inclined guiding face being opposite to a polarity of the magnetic element on each another face.

6. The trace control system for an intravenous drip as claimed in claim 1, further comprising another magnetically inductive actuator mounted on the outer periphery of the housing and diametrically opposed to the magnetically inductive actuator.

7. The trace control system for an intravenous drip as claimed in claim 1, with the shaft unit including a shaft, a ball bearing, and a waterproof washer, with the shaft coupled with the ball bearing, and with the waterproof washer mounted between the ball bearing and the housing.
